(19) United States
(12) Patent Application Publication
(10) Pub. No.: US 2024/0189477 A1
(43) Pub. Date: Jun. 13, 2024

(54) BIOSYNTHETIC DEVICES

(71) Applicant: Elastagen Pty Ltd, Eveleigh (AU)

(72) Inventors: Anthony Steven Weiss, Eveleigh (AU); Suzanne Marie Mithieux

(21) Appl. No.: 18/582,113

(22) Filed: Feb. 20, 2024

Related U.S. Application Data
(63) Continuation of application No. 16/346,053, filed on Apr. 29, 2019, now abandoned.

(30) Foreign Application Priority Data
Nov. 4, 2016 (AU) 2016904516

(51) Int. Cl. A61L 27/22; A61L 27/28; A61L 27/36; A61L 27/38; A61L 27/54; A61L 27/56; A61L 27/60; C07K 14/78

(52) U.S. Cl. CPC A61L 27/227; A61L 27/28; A61L 27/36; A61L 27/38; A61L 27/3813; A61L 27/54; A61L 27/56; A61L 27/60; C07K 14/78

(57) ABSTRACT

This invention provides a method for producing a device having elastic fiber arranged thereon. The method includes maintaining a cell culture including cells (for example, fibroblasts), cell medium and tropoelastin in conditions enabling the cells to form elastic fiber from the tropoelastin, and contacting a device with the cell culture to enable elastic fiber formed by the cells to be deposited onto the device, thereby producing a device having elastic fibers arranged thereon.

### ASSOCIATED APPLICATIONS

This application claims priority from Australian provisional application number 2016904516, the entire contents of which are hereby incorporated by reference in their entirety.

### FIELD OF THE INVENTION

The invention relates to wound healing, to matrix, scaffolds, templates, substrates and other devices and compositions for use in same, to cell culture and to elastic fiber formation.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Elastin is integral to the extracellular matrix of vertebrate tissues such as blood vessels, lungs and skin, where it provides the structural integrity and elasticity required for mechanical stretching of these tissues during normal function [1]. Elastin's three-dimensional architecture reflects its physical environment and the biological demands upon it: elastic vessels carry blood in the vasculature, the lung expands and contracts with each breath, and fibers in the dermis facilitate skin stretching and recoil.

In the dermis, elastin is arrayed in the form of fibers, the dominant component of which is the elastin polymer. There is a strong demand for de novo elastic fiber synthesis, particularly in the deep dermis, in order to maintain viable elasticity and skin function. Elastin is mainly present in the reticular portion of the dermis where large diameter elastic fibers sit deep within the tissue and are parallel to the skin surface [6].

Although elastin is one of the most durable human proteins lasting as long as the human host [2, 3] dogma states that elastic fiber synthesis in tissues including the dermis effectively ceases in early childhood [4]. After this, the regeneration of elastic fibers in full thickness wounds is severely compromised [5]. Although dermal fibroblasts are able to secrete elastin, its synthesis is repressed in the skin and many adult tissues by postitranscriptional mechanisms [7, 8].

Given above, there is an ongoing search for mechanisms that can quantitatively deliver elastic fibers into a patient's deep dermis, particularly for the repair of full thickness wounds.

Rnjak J et al 2009 [45] presents an elastic, fibrous human protein-based and cell—interactive dermal substitute scaffold based on synthetic human elastin. It describes the attachment, spreading and proliferation of fibroblasts on preformed structures or surfaces comprised of or coated with synthetic tropoelastin.

WO2013/044314 relates to utilising tropoelastin-containing compositions for elastic fibre formation in vivo.

WO2015/021508 relates to utilising tropoelastin for tissue repair.

### SUMMARY OF THE INVENTION

The invention seeks to address one or more of the above mentioned problems or limitations and in one embodiment provides a method for producing a device having elastic fiber arranged thereon. The method includes maintaining a cell culture including cells, cell medium and tropoelastin in conditions enabling the cells to form elastic fiber from the tropoelastin, and contacting a device with the cell culture to enable elastic fiber formed by the cells to be deposited onto the device, thereby producing a device having elastic fibers arranged thereon.

In another embodiment there is provided a method for producing a device including, or in the form of a collagen sheet having elastic fiber arranged thereon. The method includes maintaining a cell culture including fibroblasts, cell medium and tropoelastin in conditions enabling the fibroblasts to form elastic fiber from the tropoelastin, and contacting the collagen sheet with the cell culture to enable elastic fiber formed by the fibroblasts to be deposited onto the sheet, thereby producing a device having elastic fibers arranged thereon.

In another embodiment there is provided a method for producing a device in the form of a collagen sheet having elastic fiber arranged thereon. The method includes maintaining a cell culture including fibroblasts, cell medium and tropoelastin in conditions enabling the fibroblasts to form elastic fiber from the tropoelastin, and overlaying the collagen sheet with the cell culture to enable elastic fiber formed by the fibroblasts to be deposited onto the sheet, thereby producing a device having elastic fibers arranged thereon.

In another embodiment there is provided a method for producing a device in the form of a collagen sheet having elastic fiber arranged thereon. The method includes maintaining a cell culture including fibroblasts, cell medium and tropoelastin in conditions enabling the fibroblasts to form elastic fiber from the tropoelastin, and overlaying the collagen sheet with the cell culture so that the fibroblasts are seeded onto the collagen sheet, thereby enabling elastic fiber formed by the fibroblasts to be deposited onto the sheet, thereby producing a device having elastic fibers arranged thereon.

In any one of the above described embodiments the tropoelastin may be added to the cell medium after the device has been contacted with the cells and cell medium.

The above described embodiments may include the further step of removing the device from the cell medium to form a composition including the device and cells, or from the cell culture to obtain a device that is ostensibly cell free.

In another embodiment there is provided a device having elastic fibers arranged thereon produced by any one of the above described methods.

In another embodiment there is provided a method of forming a tissue that contains elastic fiber at a wound site including contacting a wound with a device as described above in conditions enabling healing of the wound thereby forming a tissue that contains elastic fiber at the wound site.

Further aspects of the present invention and further embodiments of the aspects described in the preceding paragraphs will become apparent from the following description, given by way of example and with reference to the accompanying figures.

### BRIEF DESCRIPTION OF THE DRAWINGS

FIG. 1: Model in vitro elastogenesis system. Elastic fiber formation by human neonatal dermal fibroblasts in the absence of exogenous tropoelastin (A) or 1(B), 3(C) and 7(D) days post tropoelastin addition. Elastic fibers (green) were stained with BA4 anti-elastin antibody and anti-mouse FITC conjugated secondary antibody. Nuclei (blue) were stained with DAPI. Images were obtained on an Olympus FluoView FV1000 confocal microscope. Scale bar=50 μm.

FIG. 2: Elastogenesis at different ages. Comparison of elastin networks formed 7 days after tropoelastin addition to cultured dermal fibroblasts sourced from different age groups—neonatal (A), 10 (B), 31 (C), 51 (D) and 92 (E) years old. Confocal microscopy images were taken of cultures stained for elastin (green) and nuclei (blue). Scale bar=50 μm.

FIG. 3: Enhanced elastogenesis through use of CM. Elastic fiber formation by dermal fibroblasts, sourced from either a 51 year old (A and B) or a neonatal (C) and cultured in FM (A and C) or CM (B) for 17 days. Tropoelastin was added on Day 10. Confocal microscopy images were taken of cultures stained for elastin (green; upper panel) and nuclei (blue; lower panel). Scale bar=50 μm. Image analysis of 10 fields of view per experiment demonstrated the enhancing effect of CM media on tropoelastin deposition (D; n=6) and fiber numbers (E; n=3) on cells sourced from a 51 year old and cultured for 17 days with tropoelastin addition on Day 10. Image analysis of the same cells grown in CM that had been divided based on the molecular weight range (<30 kDa, 30-100 kDa and >100 kDa) of its components (F). Ten fields of view were analyzed per culture medium and normalized using the average number of nuclei seen in that medium. *p<0.05; **p<0.01.

FIG. 4: Enhanced elastogenesis through multiple tropoelastin treatments. Confocal images demonstrating increasing elastin network formation with repeated tropoelastin additions to cultured dermal fibroblasts sourced from different age groups (neonatal, 10, 31 and 51 year old). Elastic fibers were not evident in untreated cultures. Cultures were stained for elastin (green) and nuclei (blue). Scale bar=20 μm.

FIG. 5: Effect of repeated tropoelastin addition on the cell matrix thickness (A) and elastic fiber content (B) of neonatal dermal fibroblasts cultured for 31 days. *p<0.05; **p<0.01; *” p<0.001; ****p<0.0001.

FIG. 6: Elastin layered cell-containing dermal substitute. Bright field and confocal images showing the capacity for an extensive elastin network layer to be formed within a dermal substitute that is cultured with both dermal fibroblasts and repeated tropoelastin treatments. Control IDRT samples cultured with only tropoelastin or cells do not exhibit an elastin network layer. H&E cross-sections show fibroblast (purple nuclei) infiltration into the IDRT increases with time. DAB-based elastin stained cross-sections show the developing elastin layer (brown stain) on the upper surface of the dermal substitute. Confocal images of this surface layer reveal an extensive elastic fiber network (orange). To distinguish between the autofluorescing collagen matrix (yellow) and the elastin network, confocal images were produced by merging images obtained through excitation at 405 nm to detect DAPI stained nuclei (blue), 488 nm to detect elastin-stained FITC fluorescence and 559 nm to detect elastin autofluorescence. Maturing elastic fibers appear orange under these conditions. H&E and elastin cross-section images scale bar=100 μm, confocal surface images scale bar=50 μm.

FIG. 7: Proposed application for full thickness wound treatment. Patient dermal fibroblasts are cultured on a dermal regeneration template where they deposit elastic fiber proteins including microfibrillar proteins and lysyl oxidases. Treatment with repeated applications of tropoelastin leads to the formation of an extensive elastic fiber network on the upper surface of the template. After it has developed the cell-matrix can be inverted and positioned within a scar tissue site.

### DETAILED DESCRIPTION OF THE EMBODIMENTS

The inventors have surprisingly found that elastic fiber that is formed on or near the cell surface of cells cultured in an in vitro cell culture system may be released from the cell surface. Further the inventors have found that the released elastic fiber may bind to a device merely by providing the device in the cell culture. Further when cells are removed from the culture, the elastic fiber remains bound to the device.

Based on these findings, the inventors have devised a method for coating a device with elastic fiber. This enables one to adapt devices, especially those indicated for treatment of full thickness wounds, so as to deliver a dense network of elastic fibers deep within the human dermis. Thus in one embodiment there is provided a method for producing a device having elastic fiber arranged thereon including:
maintaining a cell culture in a cell culture vessel, the cell culture including cells, cell medium and tropoelastin, in conditions enabling the cells to form elastic fiber from the tropoelastin; and
providing a device in the vessel, thereby contacting the device with the cell culture to enable growing cells of the culture to deposit elastic fiber formed by the cells onto the device, thereby producing a device having elastic fibers arranged thereon.

A “device” generally refers to a product that is intended for use in a tissue regeneration or tissue repair, or other therapeutic application. “Device” may refer to a scaffold, a matrix, a template, a substrate or prosthesis.

A “matrix” is generally a 3 dimensional network of synthetic and/or biological materials that may be used in tissue repair or regeneration applications, particularly in a water binding capacity, or to provide a basis for attachment of cells or therapeutic compounds. When bound to water, a matrix may form a hydrogel, which may be porous sufficient to allow the ingress or egress of cells or therapeutic compounds.

A “scaffold” is generally a 3 dimensional network of synthetic and/or biological materials that may be used in tissue repair or regeneration applications, particularly in a load bearing capacity. A scaffold may also provide for at least some of the functions of a matrix.

A “template” generally refers to a sheet or layer of synthetic and/or biological materials that may be used in tissue repair or regeneration applications, particularly for covering a wound surface. The template may be composed of a single layer, or it may be multilayered, with particular layers providing a specified function, for example moisture control. A template may be composed of cross linked networks of synthetic and/or biological molecules. The networks may form perforations, pores or slits, or alternatively, these openings or apertures may be given to a template once formed. As described herein, a particularly preferred template is a collagen based template, especially a template in which collagen is bound to a GAG (as described below).

A“substrate” generally refers to surface of a multifaceted device, such as a prosthesis or a stent.

The invention enables the production of a device having elastic fibre “arranged thereon”. It will be understood that by being “arranged thereon”, the elastic fibre may be arranged on any desired surface of the device by contact of the cell culture with same. Accordingly, where the relevant surface defines a pore or various anastomoses linking pores or other glands or chamber or passage linking same within the device, the invention enables the elastic fiber to be arranged on those relevant surfaces, and in particular those surfaces that are not immediately external facing. As explained below this can be achieved in one embodiment by immersing a device into a cell culture so that the relevant surfaces, particularly internal surfaces of the device are brought into contact with the cell culture.

In other embodiments, the device may be provided in the form of a template comprising a network of polymers having fine interstitial spaces between individual polymers and the elastic fibers are arranged in those interstitial spaces so as to ostensibly become interspersed and a part of the network of the polymers of the template.

As discussed further below, an important finding of the invention has been the determination of the fate of elastic fibre which is synthesised from tropoelastin on the cell surface. According to the invention, this fiber has been observed to be arranged on the surface of a device in the form of a collagen-containing template contained in the cell culture. By “arranged” thereon is simply meant that the fiber is ultimately deposited by growing cells on a device, so as to at least partially coat some part of the surface of the device. Accordingly it will be understood that the fiber may be deposited on, or set down on, or precipitated on to the surface of the device by growing cells during cell culture so as to at least partially coat or cover or overlay the surface.

While not wanting to be bound by hypothesis, it is believed that the elastic fiber binds to a device, principally via non covalent interactions, although it is also recognized that covalent bonds may be formed between the fiber and the device by the action of cell-derived oxidases such as lysyl oxidase, especially where the device includes a protein, for example such as collagen.

Whether non covalent or covalent interactions exist or predominate, the arrangement, or binding, or coating of the device with elastic fiber requires the contact of the cell culture with the device. In one embodiment the device may be overlaid with the cell culture, thereby contacting the device with the cell culture. In one example, the device is placed in a cell culture vessel, and the cell culture is added to the vessel so that at least one surface of the device is in contact with the cell culture. In another embodiment the device is immersed either partly or completely in the cell culture so that some or all surfaces of the device are in contact with the cell culture. This is particularly desirable where the device is porous and there is a requirement to bind elastic fiber within and about the pores of the device.

It is not necessary that the cell culture has to be completely formulated before contact with the device. For example, it is not necessary to first form a composition of cells, culture medium and tropoelastin and thereafter contact the composition with the device. In practising the invention, the tropoelastin may be added after a composition in the form of the cell culture medium and the cells have been brought into contact with the device.

Generally, the coating of the device commences after the elastic fiber has been formed on the cell surface. The rate limiting step for elastic fiber formation on the cell surface is the presence of tropoelastin. The formation of elastic fiber on the cell surface may be detected by a variety of techniques known in the art. As exemplified herein, elastic fiber formation may be detected serologically with an elastic fiber specific antibody and immunofluorescence and the quantitative and qualitative measurements of fiber production determined using publicly available software.

Factors such as the amount of tropoelastin provided in the system, the time at which it is provided, the number of cells and the surface area of the device and density of the arrangement or coating on the device are variables that determine the time in which the device should be in contact with the cell culture. Given that the cell culture is undertaken utilizing culture conditions very well understood by the skilled worker, and the assay system for measuring elastic fiber deposition on a device exemplified by the inventors herein, it is within the skill of the skilled worker to determine the contact time required to achieve a desired coating or deposition of elastic fiber on the device. An assay system for qualitative and quantitative measurement of fiber deposition on a device exemplified herein includes the use of anti-elastin antibodies and immunofluorescence microscopy and cross sectional imaging of paraffin sections of device. Cells of the cell culture are generally removed from the device before assay. If cells are lysed on the device, the fibre contained on cells (which, but for assay, may have eventually been deposited onto the device) is released onto the device. This fibre cannot be distinguished from that which has been deposited onto the device by growing cells in culture prior to assay, and this means that it is difficult to quantitate the amount of fibre that has been deposited by a growing cell in culture prior to assay.

As described above, typically the cell culture is performed in standard conditions ranging from about 5 to 10% CO₂ and about 37° C.

In one embodiment, the tropoelastin is provided in the cell culture in an amount of about 0.001 to 10 mg/ml, for example, 0.001 to 0.01, 0.005 to 0.05, 0.01 to 0.1, 0.01 to 10, 0.1 to 10 mg/ml.

Preferably the tropoelastin is in the form of SHELΔ26A as described in the examples herein.

Typically the tropoelastin is dissolved in the cell culture medium.

As described herein, the tropoelastin may be provided in the composition at the commencement of cell culture only. Alternatively, tropoelastin may be added to the cell culture at pre-determined time periods during the cell culture. In one example, the tropoelastin is given every 5 to 7 days. This latter approach ensures that an oversupply of tropoelastin drives formation of maximal amounts of elastic fiber by the cells in the culture.

Tropoelastin may be repeatedly added to the cell culture by spiking a cell culture with a tropoelastin-containing composition, or alternatively, by removing a cell culture supernatant and replacing that supernatant with fresh cell culture medium including tropoelastin.

Typically the cells are provided in the cell culture in a concentration of about 1×10³ to 1×10⁸ cells/cm² surface area, preferably 1×10⁴ to 1×10⁵ cell/cm² surface area.

In certain embodiments, it may be necessary to passage the cells during the method of the invention should the number of cells exceed a maximal amount.

The time for contact of the device with the cell culture, or in otherwords, the time of cell culture required for coating of the device with elastic fiber, may be generally about 5 to 7 days or longer. In the embodiments described herein, the cell culture was maintained for a period of 31 days. Longer or shorter periods may be required, again depending on the desired amount of coating, the amount and frequency of tropoelastin additions, and the number of cells in the cell culture.

The medium in the cell culture may be static during the period of the culture, or it may be caused to flow, for example by mechanical agitation of the culture vessel containing the cell culture. Mechanical agitation may arise by rolling, reciprocating, or shaking actions applied to the cell culture vessel.

Depending on the cell type selected for the formation of elastic fiber from the tropoelastin added to the cell culture, the cells may be seeded onto the device surface during the cell culture. In a particularly preferred embodiment, the cells may adhere to the device throughout the period of the cell culture, for example in the form of a monolayer, colony or cluster.

In other embodiments, the cells may exist in a planktonic state i.e. they may be cultured as a suspension, in which case the cells are not permanently in contact with the device for the greater period of the cell culture, although they may temporarily contact the device, for example where the cell culture is agitated causing movement of the cells.

In one embodiment, the cell culture may include a feeder layer of cells. As is known in the art, feeder cells are utilised to support the cells that are the objective of the cell culture system. For example, where the cell selected for elastic fiber formation is a stem cell, another cell type may be provided as a feeder layer for the for stem cell.

Preferably, the invention requires the addition of a tropoelastin-containing composition to the cell culture—i.e. the addition of a cell free tropoelastin-containing composition. In an alternative embodiment of the invention, a high tropoelastin-expressing cell line, for example a tropoelastin transfectant, could be utilised as a source of tropoelastin for formation of elastic fiber.

In this embodiment, the high-tropoelastin expressing cell line may additionally assemble the tropoelastin produced by it on the cell membrane to form an elastic fiber that is eventually deposited onto the device.

In a preferred embodiment a fibroblast is selected as a cell line for formation of an elastic fiber from tropoelastin added to the cell culture system. However, it will be understood that any cell or cell line capable of this function could be used for this purpose. Examples include but are not limited to cells from elastic tissues such as vascular smooth muscle cells, elastic ligament cells, lung interstitial fibroblasts, bladder smooth muscle cells, stem cells including but not limited to mesenchymal, cord blood, amniotic, embryonic and adult stem cells.

In one embodiment the method includes the further step of removing cell medium from the device, thereby producing a composition including the device having elastic fibers arranged thereon and cells of the cell culture. In this embodiment, some or all of the cells of the cell culture are retained and, depending on the use of the device, may be brought into contact with a wound site, particular at a full thickness wound. In these embodiments, it is particularly preferred that the cells of the culture, especially those selected for elastic fiber formation are ones that are not recognised as non self by the recipient of the device. In one embodiment, the cells comprised in the device are autologous or syngeneic cells, meaning that they are either derived from the individual who will ultimately receive the device, or otherwise they are tissue matched so as to have substantially the same alloantigen profile as the cells of the recipient.

In accordance with the above, in one embodiment there may be provided a method for producing a device having elastic fiber arranged thereon including:
maintaining a cell culture in a cell culture vessel, the cell culture including cells, cell medium and tropoelastin, in conditions enabling the cells to form elastic fiber from the tropoelastin;
providing a device in the vessel, thereby contacting the device with the cell culture to enable growing cells of the culture to deposit elastic fiber formed by the cells onto the device; and
removing cell medium from the device, thereby producing a composition including the device having elastic fibers arranged thereon and cells of the cell culture, thereby producing a device having elastic fibers arranged thereon.

In a separate embodiment, the method may include the further step of removing the device more or less completely from the other components of the cell culture, thereby producing an ostensibly cell free device having elastic fibers arranged thereon.

In one embodiment, the device is removed from the cell culture so as to leave the cells of the culture in the cell culture, thereby separating the cells from the device. The culture may then be reused to provide elastic fibre to a separate or different device.

In another embodiment, cells are not fixed, or lysed or killed on the device.

Another advantage of the above described embodiments that refer to cell free devices is that such a device may be used universally as it should not contain alloantigens. Elastic fibre itself is not considered to be an alloantigen. However, other components of the cells of the cell culture may be immunogenic. By removing the device from the cell culture so that the cells of the cell culture are left behind, or remain in culture, it is possible to minimise the likelihood of contamination of the device with cell derived immunogens.

In accordance with the above, in one embodiment there may be provided a method for producing a device having elastic fibre arranged thereon including:
maintaining a cell culture in a cell culture vessel, the cell culture including cells, cell medium and tropoelastin, in conditions enabling the cells to form elastic fiber from the tropoelastin;
providing a device in the vessel, thereby contacting the device with the cell culture to enable growing cells of the culture to deposit elastic fiber formed by the cells onto the device; and
removing the device from the cell culture so as to leave the cells of the culture in the cell culture, thereby separating the cells from the device, thereby producing a device having elastic fibers arranged thereon.

As discussed above, the device described herein may take the form of a scaffold, matrix or network of biological or synthetic polymers. It may also take the form of a structure having one or more impermeable inert surfaces. Such a device may be used in vivo or in vitro as a structural support for cells or tissues, enabling tissue formation, differentiation or regeneration or as a delivery system for a therapeutic. Such a device may be load bearing, bulking, filling or form a barrier or compartment within an in vivo system or device designed for use in an in vivo system.

In a particular preferred embodiment the device includes collagen, preferably collagen Type 1, although the device may also include Type II and/or III. Collagen may be derived from any source including insoluble collagen, collagen soluble in acid, in neutral or basic aqueous solutions, as well as those collagens that are commercially available. Typical animal sources for collagen include but are not limited to recombinant collagen, fibrillar collagen from bovine, porcine, ovine, cuprine and avian sources as well as soluble collagen from sources such as cattle bones and rat tail tendon.

In one preferred embodiment, the device further includes a glycosaminoglycan or GAG. GAGs are alternating copolymers made up of residues of hexosamine glycosidically bound and alternating in a more or less regular manner with either hexuronic acid or hexose moieties. Various forms of glycosaminoglycans (GAG) which may include hylauronic acid, chondroitin 6-sulfate, chondroitin 4-sulfate, heparin, heparin sulfate, keratin sulfate and dermatan sulfate.

The device may further include molecules that can be used in combination with collagen during the manufacturing process include, but are not limited to, chitin, chitosan, fibronectin, laminin, decorin, and the like, or combinations thereof.

Preferably a collagen containing device includes collagen molecules that are crosslinked and covalently bonded by a GAG as described above. The degree of cross-linking may determine the biodegradability of the device. Generally the greater the crosslink density, the lower the degradation rate and vice versa. Glutaraldehyde may be employed for cross linking collagen-GAG composites although other means for cross linking include radiation and dehydrothermal methods.

Preferably the device is biodegradable. In this embodiment, the elastic fibers may persist in the tissue after the device has degraded.

In one embodiment the collagen containing device is a template, preferably a biodegradable material with a pore size of between about 9 μm and 630 μm, a pore volume fraction of greater than about 80%, and a biodegradation rate sufficient to significantly delay or arrest the rate of wound contraction such that the time it takes a wound to contract to one-half of its original area is greater than approximately 15 days. Preferably the biodegradable material contains pores with an average size ranging from about 20 μm to about 125 μm. Preferably the biodegradable material has a degradation rate in an in vitro collagenase assay of below about 140 enzyme units, preferably below about 120 enzyme units. Preferably the collagen molecules in the template are crosslinked and covalently bonded with a glycosaminoglycan. Such a template and a manufacture process therefor is disclosed in U.S. Pat. No. 4,987,840 the contents of which are incorporated in their entirety by reference.

A researcher skilled in the art would be readily able to determine an appropriate biomaterial or mixture of biomaterials which may be utilised in the composition of the device in the current invention. The biomaterials may come from any of the typical materials used in such devices including but not limited to ceramics, synthetic polymers and natural polymers. Ceramics may include but is not limited to hydroxyapatite (HA) and tri-calcium phosphate (TCP). Synthetic polymers include but are not limited to polystyrene, poly-1-lactic acid (PLLA), polyglycolic acid (PGA), poly-dl-lactic-co-glycolic acid (PLGA) and polymethacrylates (PMAs). Natural polymers include but are not limited to extracellular matrix components such as collagens and GAGs. In addition, the device may be comprised of decellularised cadaveric or animal tissue including but not limited decellularised dermis.

Preferably the device is not glass.

In one embodiment the device may take the form of a sheet, layer or tube.

The device may be multilayered, with a first layer being a composite of a synthetic or biological polymers (such as collagen and GAG), as second layer upon one side of the first layer forming a barrier or compartment (for example a moisture controlling layer), and a third layer in the form of deposited elastic fiber upon the opposite side of the first layer. The first layer may be perforated, or it may contain pores or slits enabling the control of substances, water or gasses across the device. Examples of polymers forming the first layer include synthetic polymeric materials such as silicone polymers.

Typically a device according to the invention (i.e. a device in which elastic fibers are to be arranged or deposited thereon) is not a cell culture vessel or part thereof.

In one embodiment, the surface of the device does not comprise tropoelastin, or does not comprise synthetically cross linked tropoelastin, or synthetic elastin.

In one particularly surprising finding, the inventors have further found that fibroblasts obtained from mature aged individuals have a significantly reduced capacity to form elastic fiber in the presence of tropoelastin. Further, it has been found that cell medium conditioned by the growth of neonate fibroblasts in the medium can be used to potentiate, or accelerate, or otherwise generally increase elastic fiber production on the cell surface. Finally, it has also been found that the conditioned medium obtained from growing neonatal fibroblasts in culture can be used to increase the capacity of fibroblasts from mature age individuals to produce elastic fiber on the cell surface in the presence of tropoelastin. The latter is a particularly useful advantage because it enables elderly individuals in which there is a paucity of elastic fiber formation in full thickness wounds to utilise their own fibroblasts, in device produced by the method of the invention.

Thus in one embodiment of the invention, the cell medium is conditioned cell medium. In another embodiment the cell medium is supplemented with conditioned cell medium.

In a particularly preferred embodiment, the conditioned cell medium is conditioned by fibroblasts, preferably by neonatal fibroblasts.

The conditioned cell medium may include one or more of the proteins in Table 1 as described herein.

In another embodiment there is provided a process for increasing the production of elastic fiber by a fibroblast, the method including the step of culturing a fibroblast in a cell medium including tropoelastin, wherein the medium includes a conditioned medium obtained from the culture of a neonatal fibroblast in the medium. Preferably the fibroblast in which production of elastic fiber is to be increased is a post adolescent fibroblast, preferably and adult or mature age fibroblast.

The invention also provides a device having elastic fibers arranged thereon produced by any one of the above described methods.

Surprisingly, the inventors have found that when a porous device is placed into culture with tropoelastin and cells capable of forming elastic fibre, an elastic fiber network is formed that is 3-dimensional. Without being bound by theory, the inventors believe that the cells of the cell culture are able to penetrate the porous structure of the device and then synthesise elastic fiber thereby forming a network of fibre that is interconnected throughout the device. This finding was unexpected in view of the conventional belief that cells in culture typically grow in a 2-dimensional monolayer, even if a 3-dimensional structure is present in the cell culture dish. As such, not only is it surprising that the cells are able to migrate within the porous structure, but it is even more surprising that they are able to do this in sufficient numbers to be able to grow together within the porous device, to coacervate tropoelastin monomers, and to then produce an elastic fibre that may be interconnected throughout the porous device. This work is understood to be the first description of the production of a 3-dimensional elastic fiber network outside of the body,

The 3-dimensional network of elastic fibre that arises from fibroblast migration and tropoelastin coacervation in a 3 dimensional device is structurally different from the fiber network that is formed where fiber-producing cells are grown in monolayer in culture dishes.

In one embodiment, there is provided a method for producing a porous device having elastic fibre arranged on the surfaces of the device that define the pores of the device including the steps of: maintaining a cell culture including cells, cell medium, tropoelastin and a porous device in conditions enabling the cells to migrate into the pores of the device and to form elastic fiber on the surfaces that define the pores of the device; thereby producing the porous device having elastic fibre arranged thereon. Where the pores are connected throughout the device, the elastic fibre may be connected throughout the device. In this embodiment, the elastic fibre may deposited onto the device by growing cells, or alternatively, elastic fibre may be deposited onto the device by the action of removing cells that have migrated into, or onto the device at the completion of cell culture. The cells that may be used in this embodiment of the invention, the composition and 3 dimensional structure of the device, and culture conditions may be generally as described above. Cross-sectional images of sections of the device can be derived to assess the development of the 3-dimensional structure of the elastic fiber in cell culture.

The invention also provides a device intended for use in tissue regeneration or repair, or other therapeutic application, having elastic fibre that has been arranged on the device by a cell. In another embodiment, the invention provides a device intended for use in tissue regeneration or repair, or other therapeutic application, the device having cell synthesised elastic fibre, preferably fibroblast-synthesised elastic fibre, arranged thereon. In these embodiments, the elastic fibre is non covalently attached to the device. The elastic fibre may be provided in the form of a branched or unbranched network of fibre on the surface of the device. Preferably the elastic fibre is provided in the form of a branched network of fibre on the surface of the device. The device may or may not contain cells. The device may be constructed so as to be resorbed by tissue. In one embodiment the device is constructed from collagen.

The invention also provides a method of forming tissue containing elastic fiber at a wound site including contacting a wound with a device described above in conditions enabling healing of the wound thereby forming tissue containing elastic fiber at the wound site. Preferably the wound is a full thickness dermal wound. In other embodiments, a wound site may be in an elastic tissue such as a ligament, artery or tendon and the device is provided so as to deliver a network of elastic fibres to the wound site to enable the placement of elastic fibres in the wound site, thereby providing elasticity to the tissue, and resumption of elastic function, when the wound has healed.

In another embodiment there is provided, in a method of wound repair, the step of providing a device having elastic fibres arranged thereon to a wound. The wound may be a full thickness wound of the dermis. Typically, the device is provided to the wound for the purpose of providing cell-synthesised elastic fibre to the deep dermis of the wound, preferably to the reticular region of the dermis. In this embodiment, the device may be constructed so as to be resorbed by the tissue, or so as to be compatible with the tissue. For example, the device may be constructed of collagen. In this embodiment the wound may provided with other compounds to facilitate wound repair and/or closure.

In another embodiment there is provided a device having elastic fibres arranged thereon, preferably as produced according to an above-described method, for use in wound repair, preferably wound repair of a dermal wound, more preferably for wound repair of a full thickness dermal wound, more preferably for providing elastic fibre to the deep reticular region of a full thickness dermal wound. In still further embodiments, there is provided a device having elastic fibers arranged thereon, preferably as produced according to an above-described method, for use in wound repair including for repair of blood vessels, or for repair of wounds in organs and tissues such as lungs, or other organs where elastic fiber is required for wound repair.

It will be understood that the invention disclosed and defined in this specification extends to all alternative combinations of two or more of the individual features mentioned or evident from the text or drawings. All of these different combinations constitute various alternative aspects of the invention.

### EXAMPLES

### 1. Materials and Methods

### 1.1 Human Dermal Fibroblasts

Human dermal fibroblasts used in this study were sourced from neonatal males (NHF45C ThermoFisher; NHF8909 gift of X. Q. Wang, University of Queensland, Australia), a 10 year old male (GM03348 Coriell Institute for Medical Research), a 31 year old male (obtained from a consenting burns patient in the Burns Unit at Concord Repatriation General Hospital, NSW, Australia in accordance with the approval of the Hospital Research and Ethics Committee), a 51 year old male (142BR Sigma) and a 92 year old male (AG04064 Coriell Institute for Medical Research).

### 1.2 Tropoelastin

Recombinant human tropoelastin isoform SHELA26A (synthetic human elastin without domain 26A) corresponding to amino acid residues 27-724 of GenBank entry AAC98394 (gi 182020) was purified from bacterial culture as described previously [26, 27] (Elastagen Pty Ltd).

### 1.3 Cell Culture

### 1.3.1 Elastogenesis Model

Human dermal fibroblasts (5×10⁴ cells) were seeded on glass cover slips in the wells of 12 well tissue culture plates in Fresh Media (FM) containing DMEM (Life Technologies) with 10% (v/v) fetal bovine serum (FBS; Life Technologies) and 1% (v/v) Pen/Strep (Sigma). Cells were cultured at 37° C. 5% CO₂ and the media was changed every 2-3 days. On Day 10 of culture 1 mg tropoelastin (filter sterilized; 10 mg/ml in phosphate buffered saline (PBS)) was added to each well and the cells were cultured for a further seven days, with media changes on days 13 and 15. Control cell samples with no tropoelastin addition were cultured for 17 days. At 1, 3 or 7 days post-tropoelastin addition the cultured cells were washed twice in PBS then fixed with 4% (w/v) paraformaldehyde for 20 min and quenched with 0.2 M glycine. The cells were incubated with 0.2% (v/v) Triton X-100 for 6 min, blocked with 5% (w/v) bovine serum albumin at 4° C. overnight, and stained with a 1:500 dilution of BA4 mouse anti-elastin antibody (Sigma) for 1.5 h and a 1:100 dilution of anti-mouse IgG-FITC antibody (Sigma) for 1 h. The coverslips were mounted onto glass slides with ProLong Gold anti-fade reagent with DAPI (Invitrogen). Slides were left to set for 24 h then analyzed using a confocal microscope.

### 1.3.2 Conditioned Media

Conditioned media (CM) was prepared by collecting media from 3 day cultures of neonatal dermal fibroblasts in FM, filter sterilizing and mixing in a 1:1 ratio with DMEM containing 20% (v/v) FBS and 1% (v/v) Pen/Strep. Medium containing 20% FBS was added to account for serum components that had been depleted from the media collected from the 3 day FM cultures of neonatal fibroblasts. The final FBS concentration in the CM was up to 15%. To control for this possibility a medium containing DMEM with 15% (v/v) FBS and 1% (v/v) Pen/Strep was also tested. Fibroblasts sourced from a 51 year old male (142BR) were cultured in FM, CM or control media for 17 days with 1 mg tropoelastin (filter sterilized; 10 mg/ml in PBS) added on Day 10. Samples were fixed and stained as described above.

For size fractionation experiments CM was spun through Amicon Ultra-15 Centrifugal Filter Units (Millipore; 100 kDa and 30 kDa MWCO). Concentrated solutions of >100 kDa and 30-100 kDa were rediluted in DMEM with 10% (v/v) FBS and cells were cultured in each media as described above.

### 1.3.3 RNA Extraction

Triplicate samples of fibroblasts (1×10⁵ cells) were seeded into the wells of 6 well tissue culture plates and cultured for 11 days in FM (Neonatal and 142BR) or CM (142BR) with media changes every 2-3 days. Cells were harvested and RNA extracted using an RNeasy Mini Kit (Qiagen).

### 1.3.4 Repeated Tropoelastin Supplementation

Human dermal fibroblasts were cultured for 31 days in FM as described above. On days 10, 17 and 24 tropoelastin (1 mg filter sterilized; 10 mg/ml in PBS) was added to the wells such that the cultures were supplemented with 1, 2 or 3 additions of tropoelastin. Non-supplemented cells were also cultured. Samples were fixed and stained as described above.

### 1.3.5 Preparation of Dermal Substitute Containing Patient Cells and Elastic Fibers

IDRT (Integra Life Sciences Corporation, Plainsboro, NJ); 1.5×1.5 cm squares were placed in the wells of 12 well cell culture plates and seeded with neonatal human dermal fibroblasts (2×10⁵ cells in 200 pl FM). After 1 h at 37° C. 5% CO₂ a further 3 ml of FM was added to each well. Cells were cultured on IDRT for up to 33 days with media changes every 2-3 days. At days 12, 19 and 26 tropoelastin (1 mg filter sterilized; 10 mg/ml in PBS) was added to the wells. At days 19, 26 and 33 samples were fixed and stained following 1, 2 or 3 additions of tropoelastin. IDRT samples cultured for 33 days with cells and no tropoelastin supplementation or with no cells and 3 additions of tropoelastin were also prepared. Samples were fixed in 10% formalin. For cross-section imaging samples were embedded in paraffin, sectioned and stained with either hematoxylin and eosin or BA4 mouse anti-elastin antibody and an HRP conjugated anti-mouse secondary antibody (Dako Envision system HRP labelled polymer anti-mouse) and visualized using Liquid DAB+substrate chromogen system (Dako). A surface view was obtained using confocal microscopy of samples stained as described above.

### 1.4 RNA Analysis

For each condition, triplicate samples of RNA were probed and analyzed by microarray analysis using Affymetrix Human Prime View (U219) array at The Ramaciotti Centre for Gene Function Analysis NSW Australia. Expression Console 1.0 software (Affymetrix) was used to normalize data using RMA-sketch, which were then annotated using HuGene 1.0 ST v1 library and annotation files. Signal intensities were averaged between triplicates and SD was determined. For detection of differentially expressed genes, a p-value less than 0.05 was used in combination with a fold-change cut-off above 2.0 and signal intensity above background (i.e., 200) level. Where multiple probe sets for the same gene showed differential expression, the probe set with the largest signal intensity is reported as representative.

### 1.5 Confocal Microscopy

Fluorescently immunostained samples were visualized with an Olympus FluoView FV1000 confocal microscope using laser excitation at 405 nm to detect DAPI fluorescence, 488 nm to detect FITC fluorescence and 559 nm to detect elastin autofluorescence. Images were analyzed using ImageJ software [28]. Z-stacks were taken from 10 fields of view (FOV) per sample, converted to maximum projection images and analyzed for total area of elastic fibers and relative fiber numbers. In all cases results from 10 FOV were averaged to give a result per sample. For percent area of tropoelastin staining analysis the automated, software-generated threshold was used to exclude background pixels on each image. The number of green pixels was measured and converted to % per total area. To compare relative fiber numbers, three parallel lines were drawn and evenly distributed across each FOV. The number of fibers crossing each line was counted, added together and divided by three. The number of cell nuclei per FOV was also counted.

### 1.6 Statistics

Student's unpaired t tests (RNA analysis, relative fiber number analysis) or one-way ANOVA with Bonferroni multiple comparison tests (all other analyses) were performed using Graph Pad Prism version 6.07 software. Statistical significance was accepted at values of p<0.05. Data are presented as mean±SEM for CM and multiple tropoelastin addition experiments and mean±SD for RNA analysis. In the figures, significance is indicated by asterisks (*p<0.05, **p<0.01, ***p<0.001, ****p<0.0001).

### 2. Results and Discussion

### 2.1 Elastogenesis by Human Dermal Fibroblasts

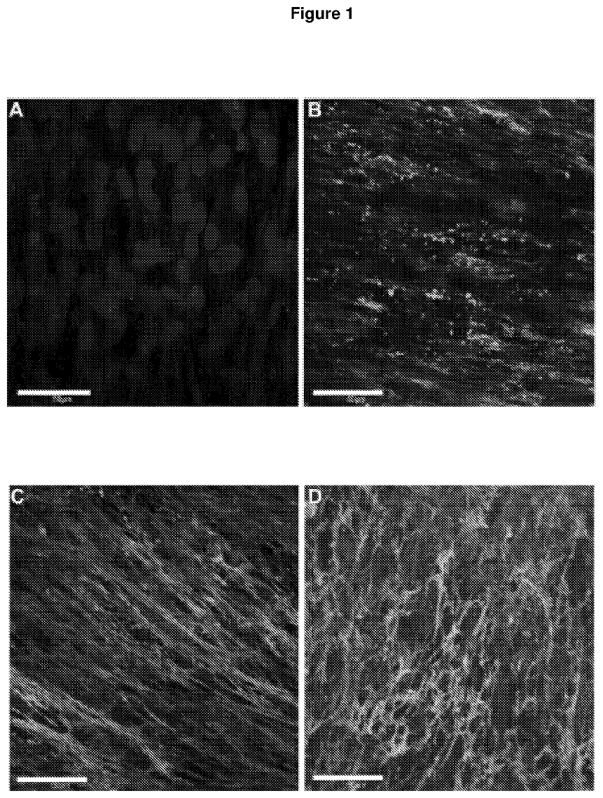

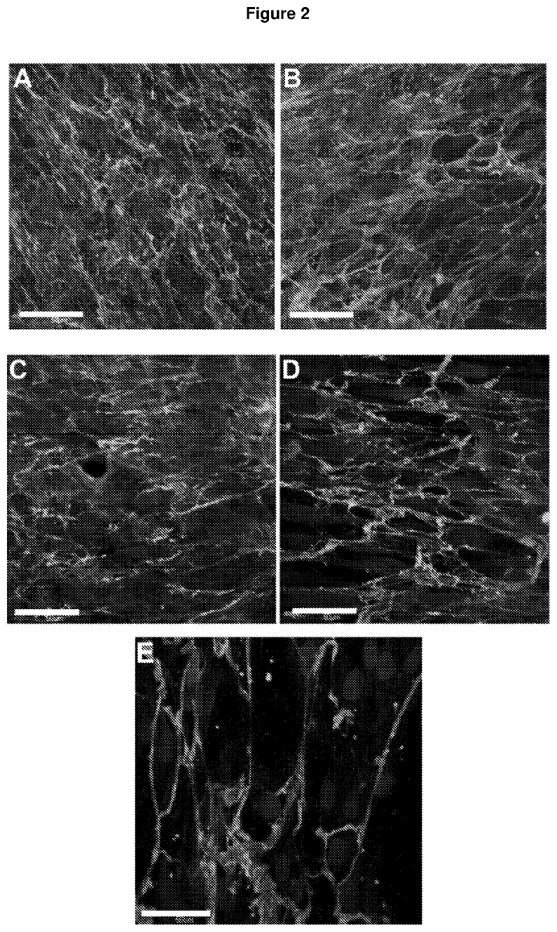

We and others [20, 29-31] have used in vitro cell culture models with the addition of recombinant tropoelastin to investigate elastogenesis by cells. In our model system, human dermal fibroblasts are cultured for 10-12 days prior to the addition of purified recombinant human tropoelastin and then cultured for up to a further 7 days. In the absence of exogenous tropoelastin no elastic fiber synthesis is evident (FIG. 1A). Following tropoelastin addition, the protein is deposited into the extracellular matrix as globules (FIG. 1B) as is also seen during normal elastogenesis [32, 33]. Subsequent fiber formation is initially aligned in the direction of cells (FIG. 1C) before an extensively branched elastic fiber network is generated (FIG. 1D). Using this model, we found that human dermal fibroblasts sourced from a wide range of donor ages (neonatal, 10, 31, 51 and 92 years old) can make elastic fibers when they are supplied with tropoelastin (FIG. 2). However the fiber architecture changes with age; cells sourced from older age groups produce fewer, thicker and less branched fibers. This indicates that dermal treatments requiring repair or replacement of damaged elastic fiber networks in older individuals may be less effective.

2.2 Enhancing Elastogenesis with CM

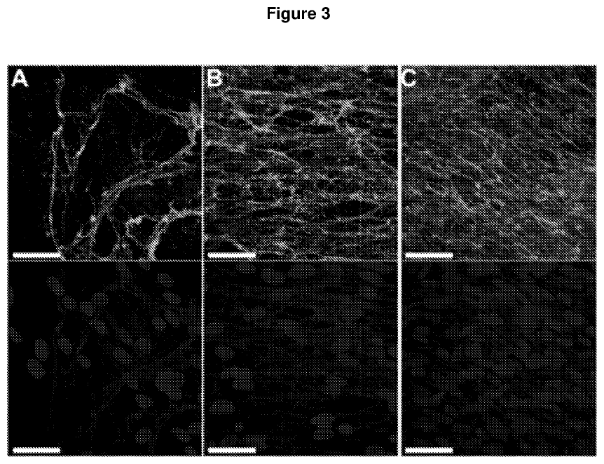

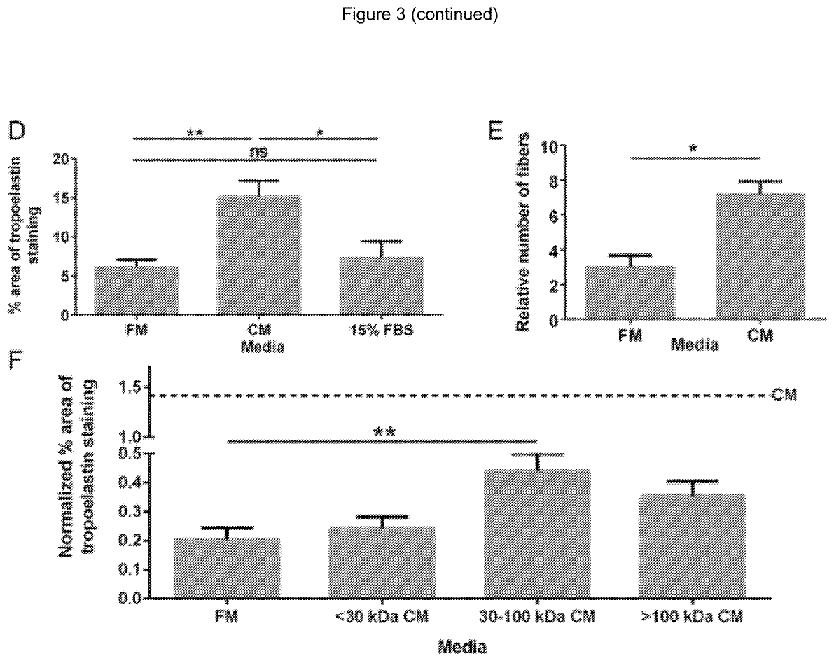

Given the ability of neonatal cells to produce extensive elastic fiber networks, we explored the effect of neonatal dermal fibroblast CM on elastogenesis. Fibroblasts were sourced from a 51-year old and treated with neonatal CM. Tropoelastin was then added to initiate elastogenesis. Compared to growth in FM (FIG. 3A), CM (FIG. 3B) resulted in a 2.5-fold increase in tropoelastin deposition into the extracellular matrix (FIG. 3D) which was accounted for by an associated 2.5-fold increase in the numbers of elastic fibers (FIG. 3E). No difference in tropoelastin deposition was seen when FM was compared with control medium (15% FBS; FIG. 3D). With the addition of CM, these older cells showed elastic networks that were comparable to those of neonatal cells (FIG. 3C). In all cases the number of nuclei per field of view was indistinguishable regardless of whether the older cells were grown in FM or CM.

Microarray analyses on triplicate samples of fibroblasts cultured for 11 days in FM (neonatal and 51 years old) or CM (51 years old) were performed to investigate the mechanism by which CM enhanced elastogenesis in older cells. Cells sourced from the 51 year old showed comparable (within 2-fold) levels of gene expression irrespective of whether they were in CM or FM, and confirmed that there was no significant change in tropoelastin expression (signal intensities 1746±228 (CM), 2060±144 (FM); p=0.113). These findings support a model where soluble factors in CM have a direct influence on the development of the elastic matrix by the older cells, rather than on gene expression. On this basis, we compared expression data from neonatal cells to older cells where both were grown in FM. Given that older cells are capable of making elastic fibers, the resulting data were filtered to only include extracellular matrix-associated proteins that were expressed by both neonatal and older cells, with a signal intensity >200, and showed statistically significant (p<0.05) increased expression levels (>2 fold) by the neonatal cells. This resulted in the identification of 7 differentially expressed genes (Table 1).

The majority of the identified targets (fibrillin 2, fibulin 1, microfibrillar associated protein 4 and latent TGFβ binding protein 1) are known elastic fiber components. Fibrillin-2 (315 kDa) predominantly regulates the early process of elastic fiber assembly [34]. It is expressed during early development with expression turned off shortly after birth. During fetal expression fibrillin 2 contributes to the microfibrillar core structure which is then overlaid postnatally by fibrillin 1[35]. Fibulin 1 (70-100 kDa) binds tropoelastin [36, 37]. Microfibrillar associated protein 4 (MFAP4; 36 kDa monomer) binds tropoelastin, desmosine, fibrillin 1 and fibrillin 2. MFAP4 promotes coacervation of tropoelastin and has been localized to the elastin-microfibril interface [38]. In support of these findings, the addition of MFAP4 to dermal fibroblast cell culture enhances elastic fiber formation with a role in the assembly of microfibrils through a proposed interaction with fibrillin 1 [39]. Latent TGFβ binding protein 1 (187 kDa) interacts with fibrillin 1 [5, 40]. Of the three remaining differentially expressed genes, thrombospondin 2 (150-160 kDa) participates in skin collagen fibrillogenesis [41], while periostin (80-90 kDa) and tenascin C (250-300 kDa) are implicated in the pathogenesis of elastofibroma dorsi, a benign fibrous soft tissue disorder characterized by an excessive number of abnormal elastic fibers [42].

It may be that a number of these factors work together to enhance elastogenesis. To test this hypothesis, the older fibroblasts were cultured in FM and supplemented with CM that had been fractionated based on molecular weight. Fractions were divided into those containing factors <30 kDa, those between 30-100 kDa, and >100 kDa. Increased elastogenesis was obtained when the 30-100 kDa fraction was independently used to supplement the FM (FIG. 3F) but did not reach the levels seen for the intact CM, which points to the involvement of multiple factors.

2.3 Enhanced Elastogenesis with Multiple Tropoelastin Treatments

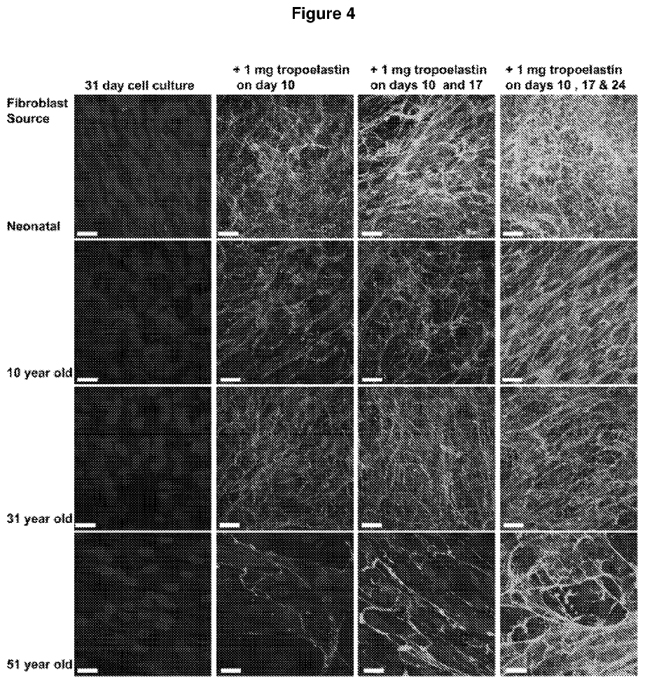

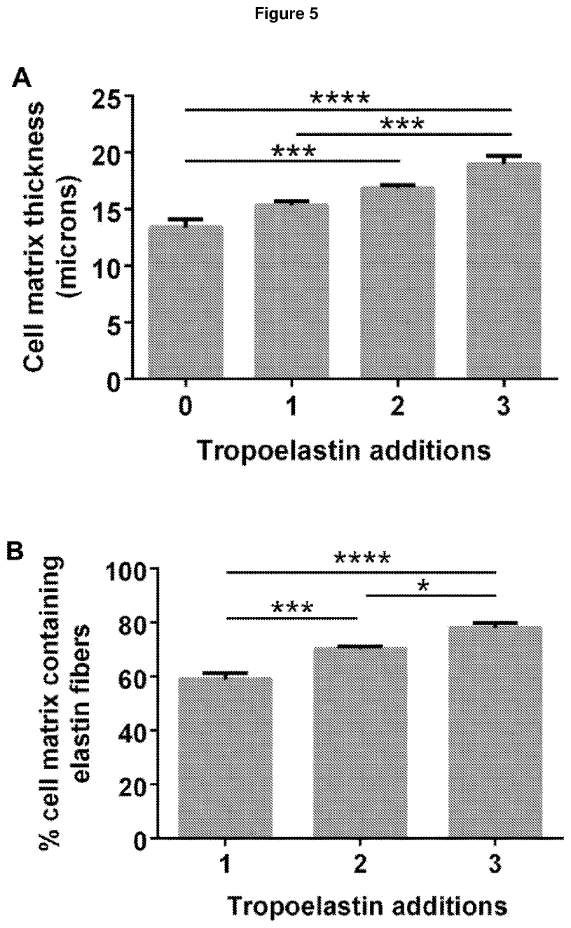

The elastogenic dependence by dermal fibroblasts on added tropoelastin was tested with multiple rounds of tropoelastin supplementation. An additional three tropoelastin treatments across a 31-day culture period demonstrated that fibroblasts from a range of age groups (0, 10, 31 and 51 year old donors) have the capacity to incorporate repeated doses of tropoelastin into a growing elastin network (FIG. 4). Regardless of the number of tropoelastin additions (0-3) the total incubation time across all samples was 31 days. In the absence of exogenous tropoelastin supplementation there was no evidence of elastic fiber synthesis which demonstrates the requirement for added tropoelastin in fiber formation. This process was accompanied by increases in cell matrix thickness that correlated with each addition of the protein. Three treatments gave rise to a 1.5-fold increase in the thickness of a neonatal dermal fibroblast culture compared to one without tropoelastin supplementation (FIG. 5A). Furthermore, the proportion of the cell matrix containing elastic fibers correspondingly increased from 59% with one tropoelastin treatment to 78% after three tropoelastin treatments (FIG. 5B).

### 2.4 Elastic Fiber Enriched Dermal Substitute

A major cause of the deficiency in elastic fiber production is the failure to upregulate tropoelastin gene expression in postnatal tissues subject to injuries. Only low maintenance levels of tropoelastin mRNA are found in most elastic tissues in adults [43] which means that there is a chronic paucity of elastin in repairing full-thickness wounds.

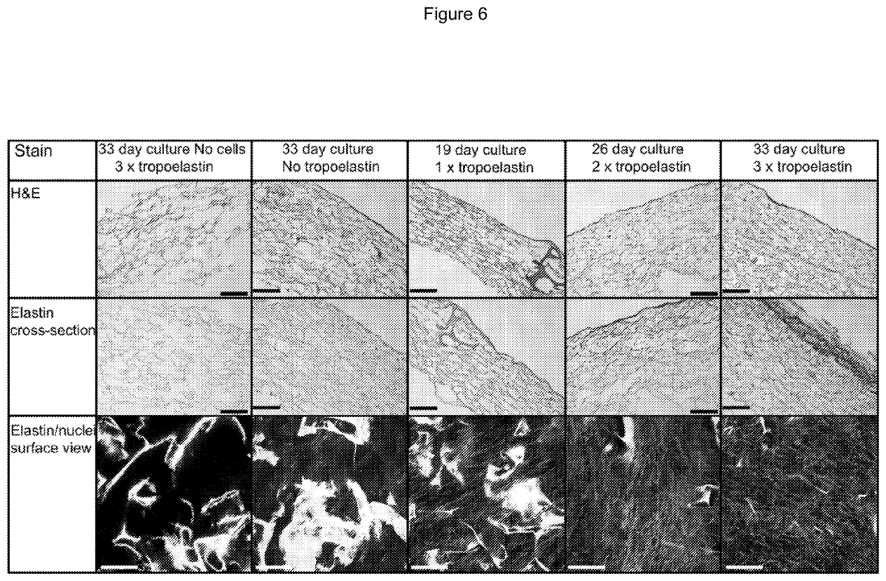

We used the technology described here to circumvent this deficiency by pre-incubating donor fibroblasts with exogenous tropoelastin on IDRT, which is the leading commercial collagen-based dermal substitute. This approach delivered elastic fibers in the upper layer, which increased with the number of doses of tropoelastin (FIG. 6). Elastin stained cross-sectional images confirmed the presence of elastin on the surface and within the scaffold. Only when cells utilize the supplemented tropoelastin do we see fibers that display both BA4 staining and intrinsic autofluorescence characteristic of elastin [21]. Elastic fibers were not evident in IDRT samples that were cultured with either cells and no tropoelastin or tropoelastin and no cells. Repeated applications of tropoelastin gave rise to a thick elastic fiber-containing layer at the top surface of the IDRT. Fluorescent elastin staining and confocal imaging confirmed the presence of an extensive network of elastic fibers in the upper layer of the IDRT, giving two effective layers: a lower IDRT region topped with a modified matrix enriched with patient elastic fibers.

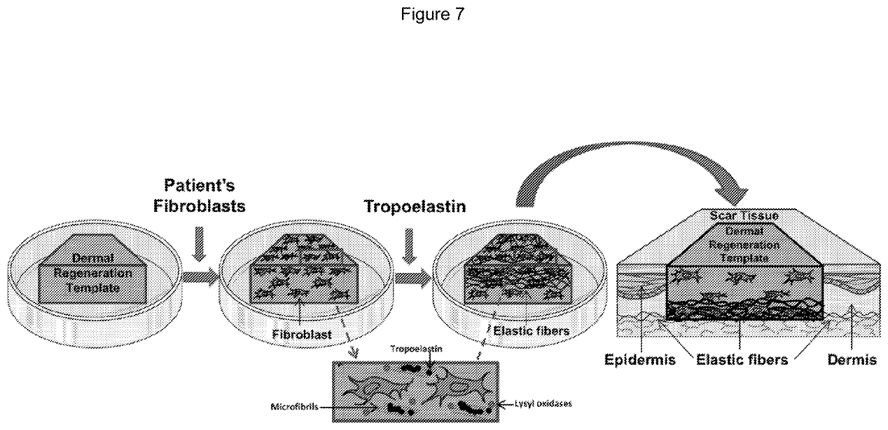

This design is attractive because it facilitates the delivery of a prefabricated elastic fiber network into the deep dermis during surgical treatment (FIG. 7). This approach is appealing because this elastic fiber network is made using autologous dermal fibroblasts and therefore comprises autologous protein components. We have previously demonstrated that recombinant human tropoelastin is well tolerated [44]. This system is designed to be compatible with human clinical use, such as revision surgery, because of its emphasis on human donor cells and synthesized human extracellular matrix.

### 3. Conclusions

We describe a process and hybrid biomaterial intended to deliver tunable levels of histologically detectable patient elastin into full-thickness wound sites. This approach addresses a persistent unmet need because repairing wounds lack this elastic substratum. Previously, dogma asserted that elastin synthesis is attenuated in early childhood but we show here that we can overcome this restriction by adding exogenous tropoelastin, regardless of the age of the dermal fibroblast donor. We describe how to further enhance synthesis by older cells by using CM. This approach delivers elastin as a layer on the leading dermal repair template for contact with the deep dermis in order to deliver prefabricated elastic fibers to the physiologically appropriate site during surgery to repair scar tissue at sites of healing full thickness wounds.

### REFERENCES

[1] Li D Y, Brooke B, Davis E C, Mecham R P, Sorensen L K, Boak B B, Eichwald E, Keating M T. Elastin is an essential determinant of arterial morphogenesis. Nature 1998; 393:276-80.
[2] Shapiro S D, Endicott S K, Province M A, Pierce J, Campbell E J. Marked longevity of human lung parenchymal elastic fibers deduced from prevalence of D-aspartate and nuclear weapons-related radiocarbon. Journal of Clinical Investigation 1991; 87:1828-34.
[3] Sivan S S, Van El B, Merkher Y, Schmelzer C E, Zuurmond A M, Heinz A, Wachtel E, Varga P P, Lazary A, Brayda-Bruno M, Maroudas A. Longevity of elastin in human intervertebral disc as probed by the racemization of aspartic acid. Biochim Biophys Acta 2012; 1820:1671-7.
[4] Kelleher C M, McLean S E, Mecham R P. Vascular extracellular matrix and aortic development. Curr Top Dev Biol 2004; 62:153-88.
[5] Raghunath M, Unsold C, Kubitscheck U, Bruckner-Tuderman L, Peters R, Meuli M. The cutaneous microfibrillar apparatus contains latent transforming growth factor-beta binding protein-1 (LTBP-1) and is a repository for latent TGF-beta1. J Invest Dermatol 1998; 111:559-64.
[6] Sherratt M J. Tissue elasticity and the ageing elastic fiber. Age 2009; 31:305-25.
[7] Langton A K, Sherratt M J, Griffiths C E, Watson R E. A new wrinkle on old skin: the role of elastic fibers in skin ageing. Int J Cosmet Sci 2010; 32:330-9.
[8] Zhang M, Pierce R A, Wachi H, Mecham R P, Parks W C. An open reading frame element mediates posttranscriptional regulation of tropoelastin and responsiveness to transforming growth factor beta1. Mol Cell Biol 1999; 19:7314-26.
[9] Rnjak J, Wise S G, Mithieux S M, Weiss A S. Severe burn injuries and the role of elastin in the design of dermal substitutes. Tissue Eng Part B Rev 2011; 17:81-91.
[10] Hirt-Burri N, Ramelet A A, Raffoul W, de Buys Roessingh A, Scaletta C, Pioletti D, Applegate L A. Biologicals and fetal cell therapy for wound and scar management. ISRN Dermatol 2011; 549870:18.
[11] Hohlfeld J, de Buys Roessingh A, Hirt-Burri N, Chaubert P, Gerber S, Scaletta C, Hohlfeld P, Applegate L A. Tissue engineered fetal skin constructs for paediatric burns. Lancet 2005; 366:840-2.
[12] Mithieux S M, Weiss A S. Elastin. Adv Protein Chem 2005; 70:437-61.
[13] Wise S G, Weiss A S. Tropoelastin. International Journal of Biochemistry & Cell Biology 2009; 41:494-7.
[14] Wagenseil J E, Mecham R P. New insights into elastic fiber assembly. Birth Defects Res C Embryo Today 2007; 81:229-40.
[15] Baldwin A K, Cain S A, Lennon R, Godwin A, Merry C L, Kielty C M. Epithelial-mesenchymal status influences how cells deposit fibrillin microfibrils. J Cell Sci 2014; 127:158-71.
[16] Sabatier L, Djokic J, Hubmacher D, Dzafik D, Nelea V, Reinhardt D P. Heparin/heparan sulfate controls fibrillin-1, -2 and -3 self-interactions in microfibril assembly. FEBS Lett 2014; 588:2890-7.
[17] Papke C L, Yanagisawa H. Fibulin-4 and fibulin-5 in elastogenesis and beyond: Insights from mouse and human studies. Matrix biology: journal of the International Society for Matrix Biology 2014; 37:142-9.
[18] Hirai M, Ohbayashi T, Horiguchi M, Okawa K, Hagiwara A, Chien K R, Kita T, Nakamura T. Fibulin-5/DANCE has an elastogenic organizer activity that is abrogated by proteolytic cleavage in vivo. J Cell Biol 2007; 176:1061-71.
[19] Wise S G, Yeo G C, Hiob M A, Rnjak-Kovacina J, Kaplan D L, Ng M K, Weiss A S. Tropoelastin: a versatile, bioactive assembly module. Acta Biomater 2014; 10:1532¬41.
[20] Yeo G C, Baldock C, Tuukkanen A, Roessle M, Dyksterhuis L B, Wise S G, Matthews J, Mithieux S M, Weiss A S. Tropoelastin bridge region positions the cell-interactive C terminus and contributes to elastic fiber assembly. Proc Natl Acad Sci USA 2012; 109:2878-83.
[21] Yeo G C, Baldock C, Wise S G, Weiss A S. A negatively charged residue stabilizes the tropoelastin N-terminal region for elastic fiber assembly. The Journal of biological chemistry 2014; 289:34815-26.
[22] Dyksterhuis L B, Carter E A, Mithieux S M, Weiss A S. Tropoelastin as a thermodynamically unfolded premolten globule protein: The effect of trimethylamine N-oxide on structure and coacervation. Arch Biochem Biophys 2009; 487:79-84.
[23] Tu Y, Weiss A S. Transient tropoelastin nanoparticles are early-stage intermediates in the coacervation of human tropoelastin whose aggregation is facilitated by heparan sulfate and heparin decasaccharides. Matrix biology: journal of the International Society for Matrix Biology 2010; 29:152-9.
[24] Yeo G C, Keeley F W, Weiss A S. Coacervation of tropoelastin. Adv Colloid Interface Sci 2011; 167:94-103.
[25] Vrhovski B, Weiss A S. Biochemistry of tropoelastin. Eur J Biochem 1998; 258:1-18.
[26] Martin S L, Vrhovski B, Weiss A S. Total synthesis and expression in Escherichia coli of a gene encoding human tropoelastin. Gene 1995; 154:159-66.
[27] Wu W J, Vrhovski B, Weiss A S. Glycosaminoglycans mediate the coacervation of human tropoelastin through dominant charge interactions involving lysine side chains. J Biol Chem 1999; 274:21719-24.
[28] Rasband W S. ImageJ. In: National Institutes of Health B, Maryland, USA, editor. http://imagejnihgov/ij/1997-2016.
[29] Kozel B A, Ciliberto C H, Mecham R P. Deposition of tropoelastin into the extracellular matrix requires a competent elastic fiber scaffold but not live cells. Matrix Biol 2004; 23:23-34.
[30] Wachi H, Sato F, Murata H, Nakazawa J, Starcher B C, Seyama Y. Development of a new in vitro model of elastic fiber assembly in human pigmented epithelial cells. Clin Biochem 2005; 38:643-53.
[31] Muiznieks L D, Miao M, Sitarz E E, Keeley F W. Contribution of domain 30 of tropoelastin to elastic fiber formation and material elasticity. Biopolymers 2016; 105:267¬75.
[32] Kozel B A, Rongish B J, Czirok A, Zach J, Little C D, Davis E C, Knutsen R H, Wagenseil J E, Levy M A, Mecham R P. Elastic fiber formation: a dynamic view of extracellular matrix assembly using timer reporters. J Cell Physiol 2006; 207:87-96.
[33] Czirok A, Zach J, Kozel B A, Mecham R P, Davis E C, Rongish B J. Elastic fiber macro-assembly is a hierarchical, cell motion-mediated process. J Cell Physiol 2006; 207:97-106.
[34] Zhang H, Hu W, Ramirez F Developmental expression of fibrillin genes suggests heterogeneity of extracellular microfibrils. J Cell Biol 1995; 129:1165-76.
[35] Charbonneau N L, Jordan C D, Keene D R, Lee-Arteaga S, Dietz H C, Rifkin D B, Ramirez F, Sakai L Y. Microfibril structure masks fibrillin-2 in postnatal tissues. J Biol Chem 2010; 285:20242-51.
[36] Kobayashi N, Kostka G, Garbe J H, Keene D R, Bachinger H P, Hanisch F G, Markova D, Tsuda T, Timpl R, Chu M L, Sasaki T. A comparative analysis of the fibulin protein family. Biochemical characterization, binding interactions, and tissue localization. J Biol Chem 2007; 282:11805-16.
[37] Sasaki T, Gohring W, Miosge N, Abrams W R, Rosenbloom J, Timpl R. Tropoelastin binding to fibulins, nidogen-2 and other extracellular matrix proteins. FEBS Lett 1999; 460:280-4.
[38] Pilecki B, Holm A T, Schlosser A, Moeller J B, Wohl A P, Zuk A V, Heumuller S E, Wallis R, Moestrup S K, Sengle G, Holmskov U, Sorensen G L. Characterization of Microfibrillar-associated Protein 4 (MFAP4) as a Tropoelastin- and Fibrillin-binding Protein Involved in Elastic Fiber Formation. J Biol Chem 2016; 291:1103-14.
[39] Kasamatsu S, Hachiya A, Fujimura T, Sriwiriyanont P, Haketa K, Visscher M O, Kitzmiller W J, Bello A, Kitahara T, Kobinger G P, Takema Y. Essential role of microfibrillar-associated protein 4 in human cutaneous homeostasis and in its photoprotection. Sci Rep 2011; 1:22.
[40] Robertson I B, Horiguchi M, Zilberberg L, Dabovic B, Hadjiolova K, Rifkin D B. Latent TGF-beta-binding proteins. Matrix Biol 2015; 47:44-53.
[41] Calabro N E, Kristofik N J, Kyriakides T R. Thrombospondin-2 and extracellular matrix assembly. Biochim Biophys Acta 2014; 8:15.
[42] Di Vito A, Scali E, Ferraro G, Mignogna C, Presta I, Camastra C, Donato G, Barni T. Elastofibroma dorsi: a histochemical and immunohistochemical study. Eur J Histochem 2015; 59.
[43] Dong X R, Majesky M W. Restoring elastin with microRNA-29: Arterioscler Thromb Vasc Biol. 2012 March; 32(3):548-51. doi: 10.1161/ATVBAHA.111.242412.
[44] Wang Y, Mithieux S M, Kong Y, Wang X Q, Chong C, Fathi A, Dehghani F, Panas E, Kemnitzer J, Daniels R, Kimble R M, Maitz P K, Li Z, Weiss A S. Tropoelastin incorporation into a dermal regeneration template promotes wound angiogenesis. Adv Healthc Mater 2015; 4:577-84.
[45] Rnjak H, Li Z, Maitz P K M, Wise S G, Weiss A S, Primary human dermal fibroblast interactions with open weave three-dimensional scaffolds prepared from synthetic human elastin Biomaterials 2009 30 6469-6477.

TABLE 1

| | | Signal | Signal | | |
|---|---|---|---|---|---|
| | | Intensity | Intensity | | |
| | Gene | 142BR cells | NHF45C cells | | Fold |
| Gene Name | Symbol | (51 year old) | (neonatal) | p value | Change |
| Fibrillin 2 | FBN2 | 598 ± 37 | 4589 ± 733 | 0.0007 | 7.7 |
| Fibulin 1 | FBLN1 | 692 ± 66 | 3819 ± 204 | <0.0001 | 5.5 |
| Microfibrillar- | MFAP4 | 297 ± 47 | 1290 ± 74 | <0.0001 | 4.3 |
| associated | | | | | |
| protein 4 | | | | | |
| Latent TGFβ | LTBP1 | 668 ± 36 | 2851 ± 291 | 0.0002 | 4.3 |
| binding | | | | | |
| protein 1 | | | | | |
| Thrombospondin | THBS2 | 1435 ± 173 | 5670 ± 1171 | 0.004 | 4.0 |
| 2 | | | | | |
| Periostin | POSTN | 2154 ± 113 | 8115 ± 226 | <0.0001 | 3.8 |
| Tenascin C | TNC | 1528 ± 247 | 4523 ± 694 | 0.002 | 3.0 |

1. A method for producing a device having elastic fiber arranged thereon including maintaining a cell culture including cells, cell medium and tropoelastin in conditions enabling the cells to form elastic fiber from the tropoelastin and contacting a device with the cell culture to enable elastic fiber formed by the cells to be deposited onto the device, thereby producing a device having elastic fibers arranged thereon.

2. The method of claim 1 wherein at least one surface of the device is overlaid with the cell culture, thereby contacting the device with the cell culture.

3. The method of claim 2 wherein the device is immersed in the cell culture.

4. The method of any one of the preceding claims wherein at least one surface of the device is contacted with cells of the cell culture.

5. The method of claim 1 wherein the device is contacted with a composition including cells and cell medium and thereafter, tropoelastin is added to the cell medium.

6. The method of claim 1 including the further step of removing the device from the cell culture, thereby producing a device having elastic fibers arranged thereon.

7. The method of claim 1 including the further step of removing cell medium from the device, thereby producing a composition including the device having elastic fibers arranged thereon and cells of the cell culture.

8. The method of any one of the preceding claims wherein the device is includes collagen.

9. The method of anyone of the preceding claims wherein the device is in the form of a sheet, layer or tube.

10. The method of any one of the preceding claims wherein the device is porous.

11. The method of claim 10 wherein the device is contacted with the cell culture to enable elastic fiber formed by the cells to be deposited in one or more pores of the device.

12. The method of any one of the preceding claims wherein the cell is a fibroblast.

13. The method of any one of the preceding claims wherein the cell medium is, or includes a conditioned cell medium.

14. The method of claim 13 wherein the conditioned cell medium is conditioned by fibroblasts.

15. The method of any one of the preceding claims wherein the cell medium includes one or more of the proteins in Table 1 as described herein.

16. A device having elastic fibers arranged thereon produced by the method of any one of the preceding claims.

17. A method of forming tissue containing elastic fiber at a wound site including contacting a wound with a device of claim 16 in conditions enabling healing of the wound thereby forming tissue containing elastic fiber at the wound site.
